SEQUENCE LISTING (18 sequences)

SEQ ID NO: 1 - DNA, 1170 bp, synthetic construct
  source 1..1170: mol_type = other DNA
atggctatagataaaagggtagttgtcctaggcacgggtgttgttggtctgtcttgcggtctggtcttgtctcgtcagggctatcgtgttcactttatcgctcgtgacttgccagaggacagtaccagccagggtttcgcgtcgccgtgggcaggcgcgaactggaccccgttttatagccgtgacgagggcccaagacaagcaaagtgggaagaagccaccttcgcacgttgggtttctctcgtgccgagcggtctggctatgtggttgaatgatacgcgtaggtacgcggataccgatgcaggtcttctgggtcattggtatcgcgacaccgtccgtaattatcgtgaactgccgccttctgagctgccgaagggtgttgcggcgggtgcggcatacgacactctgtctgtgaacgctccgctgtattgccaggcgctggcgcgtgagctgcagaccttgggtgctaccttcgagcgccgtagcgtttcaagcatcgagcaggtatttgagggtcaggacgacatcgccctggtggtgaacgccacgggtctgggtgcgaaatccatcgcggggatcgaagattccgcctgccatccggttcgtggtcaaaccgtgttggttaaaagcggctgcaaacgttgtaccatggatagcagcaatccggaagcgccggcgtacattattccgcgtccgggtggagaagttatttgcggtggcacctacctggtcgatgactgggatttgagcccgtccgcgagcacggcgcaacgtattctgacccaatgtctggcgttagatccgtcgatcagcactgacggcactctggatggcatccacattctgcgtcataacgtgggcttgcgcccagcgcgtaccggcggtccgcgcgtcgaggtgggcaaactgacccttccgctcgttcgctccacggaaccgggtactgcgcttgctctgggcaccgcgcgccctgcgccggcaggcgcgagcagcgaggcagttggtgccccgtcggaggctgtgaagcgcgaggtgaccctggttcacgcatacggcttcagctctgctggttaccagcaaagctggggcgtggcccaagacgtgctcggcctggtggaaggcgaaattggtccgccgcgcgcatggtggacccagagaggcaagctataa

SEQ ID NO: 2 - DNA, 1113 bp, synthetic construct
  source 1..1113: mol_type = other DNA
atgccaccttcagatcccataattgtactaggcgcgggcgtcatcggcctgacgaccgcggttcgtttactggaggcacacctaggcgcgaacgtgcacatcctggcggatcattggccgtccgatgccctggacgcacaatatgcgtccaccattgcaggcgctcaccacctctcgttcgccgatgacggtgatgcgcgccaacgtcgttgggatatgcgcacatttgatgttctgtacgacgaatggaaggctgttggcgagcgtactggtcttatggcattgacgcagacggaaatgtgggagggtgctacgagccatttagcggtttatgaaggcaatccggacttccgcgtactggacccgcgtaccgcaccgtgcagcaacattacccatatggttagctttaccagtctgaccattgctccgaccgtttacctggccgctctggaggctcgtgtgcgcgacctgggtgccaaactccaccgtgcccacgtgccgtctctgggcgcgcttcgcaccgacccagcgctgctggcgttgtacacccgtccgcctgcagcggtttttgtttgtgcgggtctgggtgcgcgccatctggttccggctccggaagcggcagcgttgtttccgacccgtggtcaggtcgtcgttgtccgtgcgccttggatgcgtgcaggcttcactagacaagttggtagcctgggcggcggcgagggtggcacccgtacgtacattatcccgcgttgtaatggtgaggttgtgttgggcggtaccatggaacagggtgactggactccgtacccgagagacgagaccgttaccgacatcctgacccgtgcactgcaaatttgcccggatatcgcgccaccgtatgcgcggtcttggccgaaagatgatcaggtggctgcgctgagaagcatcgtggtgcgtgacgcggtgggtttccgcccgagccgtgctggaggcgcgcgtgtcgcactggcgagcgctgcgggtatgcgagtggtgtacaactatggtcacggtggcgccggttggcagtcctgctgggggtgcgcagaagatgccgtggccctgtgggcaggtggtgcgggcggcgctcgcttgtaa

SEQ ID NO: 3 - DNA, 1026 bp, synthetic construct
  source 1..1026: mol_type = other DNA
atggatacagttaggatagctgtagtcggagcgggtgtaatgggtttgtcaaccgcagtttgtatttccaaaatggtaccggggtgctccatcaccgttatctccgacaaattcacgccggaaacaaccagcgacgtggccgcaggcatgcttatcccaccgacgtacccggataccccgattcagaagcagaagcagtggttcaaagaaacctttgaccacctgtttgcgattgtgaacagcgctgaggccgaggacgctggcgtcatcctggtttccggctggcagattttccaaagcattccgacggaggaagttccttattgggcggatgtggtcctgggttttcgtaaaatgaccaaagatgaattgaagaaattcccacagcatgtttttggtcatgcgtttacgaccctgaagtgcgaaggtccggcgtacctgccgtggctgcaaaagcgcgtgaaaggaaacggtggtctaatcctgacccgtcgtattgaggacctgtgggaactgcatccgagcttcgacatcgtggtcaactgctctggcttgggcagccgtcaactggcgggcgactcgaagatcttcccggttagaggccaggttctgaaagtgcaagcgccctgggttaaacactttatccgtgattctagcggcttgacctacatctatccgggtgtgagcaacgtgaccctgggtggcacccgccaaaagggtgattggaatctgtcgccggacgccgaaatcagcaaggagatcctctctcgttgttgcgcgctcgagccgagcctgcgtggtgcatatgatctgcgtgaaaaggtgggtctgcgtccgactcgcccatctgttcgtctggagaaagaattgctggcgcaggattcgagacgtttaccggtcgtgcaccactacggccacggcagcggtggtattgcaatgcattggggtactgccttggaggcgacccgccttgttaatgagtgcgttcaagttctgcgcaccccggctccgaaaagcaagctgtaa

SEQ ID NO: 4 - DNA, 969 bp, synthetic construct
  source 1..969: mol_type = other DNA
atgcccaaaatagcagtactaggagctgggatcaacggcatcgccagcgcgcttgccattcaagaacgtcttccgaactgcgaagttaccatcatcgcggagaaattcagcccgaacaccacctccgatgtggcggcaggcctgattgaaccatttctgtgtgatgacgacgtggatcgtatcatcaattggacctctgccaccatcagccgcatccacgaataccaggcggatggtaatccgggtgcagaagagcagagcggctactggctccaaagcgttaaaagcgaaccgaagtggttgaagctgatgaaaaacgtgcacattctaactgacgcggagatgaaacaagtagctcgccgtcctgaacataaattcggcattttttataccacgtggtacctggaaccgactccgtatattaaatggtgtaccgacaagttcctgaaaaacggtggcaaattcaagaaacagaaaattgagaacatcgatgacgtcgcgcgcagctatgatgtgaccgttaattgcaccggtctgggcagccgcgctctgattggtgataaagaggtttacccgacgcgtggtcaaattttgaaggtgagctgcccgcgtgttaagcactttttcatcgacgacaagtattacgctttactgaatgatagcaccattaccttgggcggcacgtttgaagcacatcagtgggacctgacgatcaactccgagctgtcacagaaaattctcaaggagaatatccacaatatcccgtcgctgcgtaccgcgcagattttgtcttcccacgtggacatgcgtccgagcagaggcacggttcgtttgcaagcggagctggggcgttcgctggttcacaactatggtcatggtggttctggtattaccctgcattggggttgcgcgttggagtgcgctgagatcgtggaaaacgtcctgaagatgaagaagtccaaactgtaa

SEQ ID NO: 5 - DNA, 1002 bp, synthetic construct
  source 1..1002: mol_type = other DNA
atgacacccaaaattgctataataggagaaggcgtcatcggctgctccaccgcactgcaggttgcgcaggcggtgcctgatgcacgcgtgacggttttgtctgaccgtccgtttgaacagacctgttcttttggaccggctggtctgttccgcattgatgatattgcgaaccgtgagttcggcaagagcaccttcgactggttcgcccacctgcatcgtaccgaaaagggtgacaaaaccggcgtgaagttgctgagcggtcatattcaaagcgattccaaagaacgtctggagcagcaacaaaaagcgtatggtgacattgtgtacaattttcgtttcctggagaagcgcgagatcctcgacctgttcccgaatccgagcgagcactgcatccactacactgcgtttgcgagcgaaggcaacaaatacgttccgtatctgaaattccagtgtcaggcacgtggcgtggagtttctgcacagaaaggtgcgtgatttggaggaactggcgaacgaaggttatgatgtgatcgtgaactgcgcaggcttatccggcggtacgctggcgggcgacgacgacagcgtctacccgattcgtggtgttgtcctggacgttgaagcccattggcacaaacacttcaactataaagattttatcaccttcaccattccgaaagagaactctgtagttattggcagcgtgaaacaagaaaaccgctgggatttggagatcaccgatgttgaccgcaaggacatcttggagcgctatgtggccctgcatccggctatgcgtgaaccaaaaatcctgggtgaatggtcaggtctgcgtccggctcgtaagacgatccgtattgaaaaagttgagaagaagtcggaaaagtcgggcaaaaagtacactgttgtccaccactacggccatggtggtaatggttttaccctcggttggggtacagccgtggaggctaccaaattagttaagtccgcgctgaatagcagcaagctt

SEQ ID NO: 6 - DNA, 1320 bp, synthetic construct
  source 1..1320: mol_type = other DNA
atgcatctgcgttttccgaccaccaccacctatagtctgcgcccgagcagcacccgcaccagccctcctcctctgattgttagctttgccccgcgccatagcattgtgagttataccaccagtccgcgccgcctgtttagtattattatgagcgaaccgaaaggccatgtggttgttattggcgcaggcgtgattggtctgagcagcgccctggcactgctggaagcaaattatgcaaccaccattctggccaaagatctgccggcaccgtttgaaagtattgatccgcgtagccagattaattttaccagcccgtggggtggtgcacataatcgttgggtgccgccgtttccgccgagtagtagtagcagcagtaccccgctgaccccgacccagcaggcagaacatgcactgcgtattcgtgaacatgcatttagcctgagcacctttcatcgtatgcagctgtttcagagccagggtcaggatcagcaggcaggcattacctttctgaaaggcattgaatatctggaaagcccgggcccggaatataccagcctgaccccgcagcgtgccagtcaggaactgggcctgccgggtcattttcgtgtgctggaaacccatgaatttccggatgataaagttcagtggggttgcgaatatgatacctggtgcgtgaatccgatgcagtattgcctgtttctgctgggtcagattattgcacgcggtggtaaagtgtttaaacgtgatgtgcgtagcgttgcagaagtgtttcagctgtttagtgatagcgttcaggaatttggtgccaccattccgccggccgatgcagtggtgaatgcaaccggtattggcctgggcgatgatgaaatggtgtttccgacccgtggtcagacctgcctggtgcaggaaccgtgcgatgcaaccgtgacccgccagaatgccgatggtacctggaccttttgtgtgccgcgtggctttaaagcaggtaccattattggcggcaccaaagaaccggataattgggatccgaaaccggatccggaagttcgtgaacgtctgctgcgtgcctttgaaggtacctatccgcgtattctggccgatggcaaaacccgcctgaccccggtgcgcgatattgtgggtcgtcgtccgacccgcaaaggtggcctgcgtctggaaggcgaagtggttgatggtgccggttttgtgatgcatgcctatggcctgggcggtcgtggttatgaactgagttggggtgttgccgaaggtgttattgaaggcattcagggtcatctggaaagcgaaaaaggcagtcgtctgtaa

SEQ ID NO: 7 - DNA, 294 bp, synthetic construct
  source 1..294: mol_type = other DNA
atgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaa

SEQ ID NO: 8 - DNA, 1647 bp, synthetic construct
  source 1..1647: mol_type = other DNA
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 9 - DNA, 1917 bp, synthetic construct
  source 1..1917: mol_type = other DNA
atgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataa

SEQ ID NO: 10 - DNA, 1131 bp, synthetic construct
  source 1..1131: mol_type = other DNA
atggctaagcaagattattacgagattttaggcgtttccaaaacagcggaagagcgtgaaatcagaaaggcctacaaacgcctggccatgaaataccacccggaccgtaaccagggtgacaaagaggccgaggcgaaatttaaagagatcaaggaagcttatgaagttctgaccgactcgcaaaaacgtgcggcatacgatcagtatggtcatgctgcgtttgagcaaggtggcatgggcggcggcggttctggcggcggcgcagacttcagcgatatttttggtgacgttttcggcgatatttttggcggcggacgtggtcgtcaacgtgcggcgcgcggtgctgatttacgctataacatggagctcaccctcgaagaagctgtacgtggcgtgaccaaagagatccgcattccgactctggaagagtgtgacgtttgccacggtagcggtgcaaaaccaggtacacagccgcagacttgtccgacctgtcatggttctggtcaggtgcagatgcgccagggattcttcgctgtacagcagacctgtccacactgtcagggccgcggtacgctgatcaaagatccgtgcaacaaatgtcatggtcatggtcgtgttgagcgcagcaaaacgctgtccgttaaaatcccggcaggggtggacactggagaccgcatccgtcttgcgggcgaaggtgaagcgggcgagcatggcgcaccggcaggcgatctgtacgttcaggttcaggttaaacagcacccgattttcgagcgtgaaggcaacaacctgtattgcgaagtcccgatcaacttcgctatggcggcgctgggtggcgaaatcgaagtaccgacccttgatggtcgcgtcaaactgaaagtgcctggcgaaacccagaccggtaagctattccgtatgcgcggtaaaggcgtcaagtctgtccgcggtggcgcgcagggtgatttgctgtgccgcgttgtcgtcgaaacaccggtaggcctgaacgaaaggcagaaacagctgctgcaagagctgcaagaaagcttcggtggcccaaccggcgagcacaacagcccgcgctcaaagagcttctttgatggtgtgaagaagttttttgacgacctgacccgctaa

SEQ ID NO: 11 - DNA, 594 bp, synthetic construct
  source 1..594: mol_type = other DNA
atgagtagtaaagaacagaaaacgcctgaggggcaagccccggaagaaattatcatggatcagcacgaagagattgaggcagttgagccagaagcttctgctgagcaggtggatccgcgcgatgaaaaagttgcgaatctcgaagctcagctggctgaagcccagacccgtgaacgtgacggcattttgcgtgtaaaagccgaaatggaaaacctgcgtcgtcgtactgaactggatattgaaaaagcccacaaattcgcgctggagaaattcatcaacgaattgctgccggtgattgatagcctggatcgtgcgctggaagtggctgataaagctaacccggatatgtctgcgatggttgaaggcattgagctgacgctgaagtcgatgctggatgttgtgcgtaagtttggcgttgaagtgatcgccgaaactaacgtcccactggacccgaatgtgcatcaggccatcgcaatggtggaatctgatgacgttgcgccaggtaacgtactgggcattatgcagaagggttatacgctgaatggtcgtacgattcgtgcggcgatggttactgtagcgaaagcaaaagcttaa

SEQ ID NO: 12 - DNA, 1299 bp, synthetic construct
  source 1..1299: mol_type = other DNA
atgcaagtttcagttgaaaccactcaaggccttggccgccgtgtaacgattactatcgctgctgacagcatcgagaccgctgttaaaagcgagctggtcaacgttgcgaaaaaagtacgtattgacggcttccgcaaaggcaaagtgccaatgaatatcgttgctcagcgttatggcgcgtctgtacgccaggacgttctgggtgacctgatgagccgtaacttcattgacgccatcattaaagaaaaaatcaatccggctggcgcaccgacttatgttccgggcgaatacaagctgggtgaagacttcacttactctgtagagtttgaagtttatccggaagttgaactgcagggtctggaagcgatcgaagttgaaaaaccgatcgttgaagtgaccgacgctgacgttgacggcatgctggatactctgcgtaaacagcaggcgacctggaaagaaaaagacggcgctgttgaagcagaagaccgcgtaaccatcgacttcaccggttctgtagacggcgaagagttcgaaggcggtaaagcgtctgatttcgtactggcgatgggccagggtcgtatgatcccgggctttgaagacggtatcaaaggccacaaagctggcgaagagttcaccatcgacgtgaccttcccggaagaataccacgcagaaaacctgaaaggtaaagcagcgaaattcgctatcaacctgaagaaagttgaagagcgtgaactgccggaactgactgcagaattcatcaaacgtttcggcgttgaagatggttccgtagaaggtctgcgcgctgaagtgcgtaaaaacatggagcgcgagctgaagagcgccatccgtaaccgcgttaagtctcaggcgatcgaaggtctggtaaaagctaacgacatcgacgtaccggctgcgctgatcgacagcgaaatcgacgttctgcgtcgccaggctgcacagcgtttcggtggcaacgaaaaacaagctctggaactgccgcgcgaactgttcgaagaacaggctaaacgccgcgtagttgttggcctgctgctgggcgaagttatccgcaccaacgagctgaaagctgacgaagagcgcgtgaaaggcctgatcgaagagatggcttctgcgtacgaagatccgaaagaagttatcgagttctacagcaaaaacaaagaactgatggacaacatgcgcaatgttgctctggaagaacaggctgttgaagctgtactggcgaaagcgaaagtgactgaaaaagaaaccactttcaacgagctgatgaaccagcaggcgtaa

SEQ ID NO: 13 - protein, 389 aa, synthetic construct
MAIDKRVVVLGTGVVGLSCGLVLSRQGYRVHFIARDLPEDSTSQGFASPWAGANWTPFYSRDEGPRQAKWEEATFARWVSLVPSGLAMWLNDTRRYADTDAGLLGHWYRDTVRNYRELPPSELPKGVAAGAAYDTLSVNAPLYCQALARELQTLGATFERRSVSSIEQVFEGQDDIALVVNATGLGAKSIAGIEDSACHPVRGQTVLVKSGCKRCTMDSSNPEAPAYIIPRPGGEVICGGTYLVDDWDLSPSASTAQRILTQCLALDPSISTDGTLDGIHILRHNVGLRPARTGGPRVEVGKLTLPLVRSTEPGTALALGTARPAPAGASSEAVGAPSEAVKREVTLVHAYGFSSAGYQQSWGVAQDVLGLVEGEIGPPRAWWTQRGKL

SEQ ID NO: 14 - protein, 370 aa, synthetic construct
MPPSDPIIVLGAGVIGLTTAVRLLEAHLGANVHILADHWPSDALDAQYASTIAGAHHLSFADDGDARQRRWDMRTFDVLYDEWKAVGERTGLMALTQTEMWEGATSHLAVYEGNPDFRVLDPRTAPCSNITHMVSFTSLTIAPTVYLAALEARVRDLGAKLHRAHVPSLGALRTDPALLALYTRPPAAVFVCAGLGARHLVPAPEAAALFPTRGQVVVVRAPWMRAGFTRQVGSLGGGEGGTRTYIIPRCNGEVVLGGTMEQGDWTPYPRDETVTDILTRALQICPDIAPPYARSWPKDDQVAALRSIVVRDAVGFRPSRAGGARVALASAAGMRVVYNYGHGGAGWQSCWGCAEDAVALWAGGAGGARL

SEQ ID NO: 15 - protein, 341 aa, synthetic construct
MDTVRIAVVGAGVMGLSTAVCISKMVPGCSITVISDKFTPETTSDVAAGMLIPPTYPDTPIQKQKQWFKETFDHLFAIVNSAEAEDAGVILVSGWQIFQSIPTEEVPYWADVVLGFRKMTKDELKKFPQHVFGHAFTTLKCEGPAYLPWLQKRVKGNGGLILTRRIEDLWELHPSFDIVVNCSGLGSRQLAGDSKIFPVRGQVLKVQAPWVKHFIRDSSGLTYIYPGVSNVTLGGTRQKGDWNLSPDAEISKEILSRCCALEPSLRGAYDLREKVGLRPTRPSVRLEKELLAQDSRRLPVVHHYGHGSGGIAMHWGTALEATRLVNECVQVLRTPAPKSKL

SEQ ID NO: 16 - protein, 322 aa, synthetic construct
MPKIAVLGAGINGIASALAIQERLPNCEVTIIAEKFSPNTTSDVAAGLIEPFLCDDDVDRIINWTSATISRIHEYQADGNPGAEEQSGYWLQSVKSEPKWLKLMKNVHILTDAEMKQVARRPEHKFGIFYTTWYLEPTPYIKWCTDKFLKNGGKFKKQKIENIDDVARSYDVTVNCTGLGSRALIGDKEVYPTRGQILKVSCPRVKHFFIDDKYYALLNDSTITLGGTFEAHQWDLTINSELSQKILKENIHNIPSLRTAQILSSHVDMRPSRGTVRLQAELGRSLVHNYGHGGSGITLHWGCALECAEIVENVLKMKKSKL

SEQ ID NO: 17 - protein, 334 aa, synthetic construct
MTPKIAIIGEGVIGCSTALQVAQAVPDARVTVLSDRPFEQTCSFGPAGLFRIDDIANREFGKSTFDWFAHLHRTEKGDKTGVKLLSGHIQSDSKERLEQQQKAYGDIVYNFRFLEKREILDLFPNPSEHCIHYTAFASEGNKYVPYLKFQCQARGVEFLHRKVRDLEELANEGYDVIVNCAGLSGGTLAGDDDSVYPIRGVVLDVEAHWHKHFNYKDFITFTIPKENSVVIGSVKQENRWDLEITDVDRKDILERYVALHPAMREPKILGEWSGLRPARKTIRIEKVEKKSEKSGKKYTVVHHYGHGGNGFTLGWGTAVEATKLVKSALNSSKL

SEQ ID NO: 18 - protein, 439 aa, synthetic construct
MHLRFPTTTTYSLRPSSTRTSPPPLIVSFAPRHSIVSYTTSPRRLFSIIMSEPKGHVVVIGAGVIGLSSALALLEANYATTILAKDLPAPFESIDPRSQINFTSPWGGAHNRWVPPFPPSSSSSSTPLTPTQQAEHALRIREHAFSLSTFHRMQLFQSQGQDQQAGITFLKGIEYLESPGPEYTSLTPQRASQELGLPGHFRVLETHEFPDDKVQWGCEYDTWCVNPMQYCLFLLGQIIARGGKVFKRDVRSVAEVFQLFSDSVQEFGATIPPADAVVNATGIGLGDDEMVFPTRGQTCLVQEPCDATVTRQNADGTWTFCVPRGFKAGTIIGGTKEPDNWDPKPDPEVRERLLRAFEGTYPRILADGKTRLTPVRDIVGRRPTRKGGLRLEGEVVDGAGFVMHAYGLGGRGYELSWGVAEGVIEGIQGHLESEKGSRL

(19) United States
(12) Patent Application Publication
(10) Pub. No.: US 2026/0242750 A1
(43) Pub. Date: Aug. 20, 2026

(54) METHOD FOR EXPRESSING D-AMINO ACID OXIDASE AND USE THEREOF

(71) Applicants: ZHEJIANG WYNCA CHEMICAL INDUSTRY GROUP CO., LTD, Hangzhou, Zhejiang (CN); ZJU-HANGZHOU GLOBAL SCIENTIFIC AND TECHNOLOGICAL INNOVATION CENTER, Hangzhou, Zhejiang (CN)

(72) Inventors: Shuguang ZHOU, Hangzhou, Zhejiang (CN); Lirong YANG, Hangzhou, Zhejiang (CN); Long QIN, Hangzhou, Zhejiang (CN); Jiaqi XU, Hangzhou, Zhejiang (CN); Shenluan YU, Hangzhou, Zhejiang (CN); Haisheng ZHOU, Hangzhou, Zhejiang (CN); Xin KANG, Hangzhou, Zhejiang (CN); Ziyuan WANG, Hangzhou, Zhejiang (CN); Yiyuan LOU, Hangzhou, Zhejiang (CN); Zewei ZHANG, Hangzhou, Zhejiang (CN)

(73) Assignees: ZHEJIANG WYNCA CHEMICAL INDUSTRY GROUP CO., LTD, Hangzhou, Zhejiang (CN); ZJU-HANGZHOU GLOBAL SCIENTIFIC AND TECHNOLOGICAL INNOVATION CENTER, Hangzhou, Zhejiang (CN)

(21) Appl. No.: 19/162,244

(22) PCT Filed: Mar. 29, 2023
(86) PCT No.: PCT/CN2023/084680

§ 371 Date: Sep. 4, 2025

(30) Foreign Application Priority Data
Mar. 9, 2023 (CN) 202310221079.X

(51) Int. Cl. C12N 9/06; C12N 15/70; C12P 7/40

(52) U.S. Cl. CPC C12N 9/0024; C12N 15/70; C12P 7/40; C12Y 104/03003

(57) ABSTRACT

Provided are a method for expressing a D-amino acid oxidase and the use thereof. A wild type D-amino acid oxidase having catalytic activity on catalyzing a substrate, DL-glufosinate-ammonium, is obtained by screening; and the activity and soluble expression of the D-amino acid oxidase obtained by host expression are further improved by means of expression host screening, addition of molecular chaperones and the like. The present invention achieves kinetic resolution of racemic glufosinate-ammonium, and can be used for production of L-glufosinate-ammonium.

This application claims the priority of Chinese Patent Application No. 202310221079.X, filed with the China National Intellectual Property Administration on Mar. 9, 2023, and titled with “METHOD FOR EXPRESSING D-AMINO ACID OXIDASE AND USE THEREOF”, the disclosure of which is hereby incorporated by reference in its entirety.

### FIELD

The present disclosure relates to the technical field of biochemical engineering, and in particular to a method for expressing a D-amino acid oxidase and use thereof.

### BACKGROUND

D-amino acid oxidase (DAAO, EC 1.4.3.3) is a flavoprotein containing flavin adenine dinucleotide (FAD). Under aerobic conditions, this enzyme can catalyze the enantioselective oxidation and dehydrogenation of D-amino acids to produce imidic acid. The imidic acid undergoes spontaneous hydrolysis to generate the corresponding α-keto acid and ammonia. During this reaction, the reduced coenzyme FADH₂ is re-oxidized by oxygen to produce hydrogen peroxide.

So far, the reported DAAOs are mainly derived from eukaryotic organisms, for example filamentous fungi, yeasts and mammals. Among them, typical DAAOs include pkDAAO from porcine kidney, RgDAAO from Rhodotorula, and TvDAAO from Trigonopsis variabilis. In addition, DAAOs have been successively found in prokaryotic organisms of the genus Actinomycetes, e.g., ApDAAO derived from Arthrobacter protophormiae. These DAAOs of various sources not only play an important role in the metabolism of D-amino acids, but also lay a foundation for the application of this class of enzymes in biotechnology.

DAAOs typically have a broad substrate spectrum and highly enantioselective characteristic, and have been successfully used in a variety of biochemical reaction processes. The related studies have attracted extensive attention from scholars at home and abroad. Over the years, DAAOs are used in biocatalysis, mainly in the synthesis of 7-aminocephalosporanic acid, chiral amino acids, and α-keto acids. Among them, α-keto acids are an important raw material of chemical industrial, and synthesizing α-keto acids in a green and efficient way has been a hot research topic in recent years. DAAO-catalyzed oxidative dehydrogenation of D-amino acids is an important pathway for the synthesis of the corresponding α-keto acids. For example, some studies have reported a method of producing α-keto acids by using racemic amino acids as substrates, conversing L-amino acids into D-amino acids by the amino acid racemase BsrV derived from Vibrio cholerae, and converting D-amino acids to α-keto acids by TvDAAO, with a conversion rate of more than 99%. This method offers advantages of high reaction efficiency and environmental friendliness, while avoiding the use of expensive D-amino acids as raw materials.

Phosphinothricin (also known as glufosinate) is a phosphorus-containing amino acid sterilant herbicide, chemically known as 2-amino-4-(hydroxymethyl phosphonyl)butanoic acid, which has the characteristics of wide herbicidal spectrum, low toxicity, long effective duration, high activity, good environmental compatibility, etc., and is widely used in prevention and control of weeds in farmland crops, non-cultivated land, no-tillage land and the like. Among two enantiomers of glufosinate: L-glufosinate (active glufosinate ammonium) and D-glufosinate, only the L-glufosinate has herbicidal activity. Currently, all market-selling glufosinate is racemic. If an optically pure L-glufosinate is prepared for application, the atom economy will be significantly improved and the environment pressure will be reduced. D-amino acid oxidase can oxidize D-glufosinate in the racemic glufosinate to the corresponding α-keto acid, which is further converted to the L-glufosinate by L-amino acid dehydrogenase or transaminase. This process theoretically has certain application potential, but the existing problem is the lack of genetic sources of D-amino acid oxidase—the sufficiently efficient enzyme catalyst and an efficient soluble expression system of D-amino acid oxidase.

### SUMMARY

In view of this, the technical problem to be solved by the present disclosure is to provide a method for expressing a D-amino acid oxidase and use thereof. The present disclosure provides a method for expressing each of 6 wild-type D-amino acid oxidase genes in a host, and use of the D-amino acid oxidases in the production of a α-keto acid.

The present disclosure provides a nucleic acid encoding a D-amino acid oxidase, wherein the nucleic acid comprises at least one sequence selected from the group consisting of:
(I) a nucleotide sequence set forth in any one of SEQ ID NOs: 1 to 6,
(II) a nucleotide sequence encoding the same protein as the nucleotide sequence in (I), but different from the nucleotide sequence in (I) due to the degeneracy of genetic codon,
(III) a nucleotide sequence derived from the nucleotide sequence in (I) or (II) by substitution, deletion or addition of one or more nucleotides, and having an identical or similar function to the nucleotide sequence in (I) or (II), and
(IV) a nucleotide sequence having at least 90% sequence homology to the nucleotide sequence in (I), (II) or (III).

In the present disclosure, the nucleic acid may be DNA, RNA, cDNA or PNA. In an embodiment of the present disclosure, the nucleic acid is in the form of DNA. The DNA form includes cDNA, genomic DNA or artificially synthetic DNA. The DNA may be single stranded or double stranded. The nucleic acid may comprise nucleotide sequences that have different functions, for example, a coding region and a non-coding region such as a regulatory sequence (e.g., a promoter or a transcription terminator). The RNA form is mRNA obtained by gene transcription or the like.

In the present disclosure, 27 nucleic acids encoding D-amino acid oxidases from various sources were collected, constructed into plasmid vectors and expressed in hosts. The enzyme activity of D-amino acid oxidase on a substrate was detected to screen the D-amino acid oxidases. The results showed that six D-amino acid oxidases had enzyme activity on the substrate DL-glufosinate, and they are from Rhodotorula graminis WP1, Vanrija humicola, Bos taurus, Caenorhabditis elegans and Neurospora crassa OR74A. Subsequently, these six nucleic acids encoding D-amino acid oxidases were subjected to codon optimization for Escherichia coli, and the optimized nucleic acids were found to have a high expression level in the host and an increased soluble expression and the obtained D-amino acid oxidases showed high enzyme activity. In an embodiment of the present disclosure, the sequences of six optimized nucleic acids encoding D-amino acid oxidases are set forth in SEQ ID NOs: 1 to 6. These six fragments are capable of expressing D-amino acid oxidase more efficiently relative to other codon-optimized fragments. For example, SEQ ID NO: 1 is an optimized sequence encoding D-amino acid oxidase 1 (DAAO1) derived from Rhodotorula graminis WP1. After transformation into the host, the sequence of SEQ ID NO: 1 is capable of exhibiting a higher expression to afford higher enzyme activity compared to other optimized nucleic acids encoding DAAO1.

The present disclosure provides an expression cassette comprising a promoter, any nucleic acid encoding a D-amino acid oxidase of the present disclosure, and a terminator.

Further, the pression cassette includes an expression module comprising the nucleic acid of the present disclosure in single or multiple tandem form with the promoter and terminator, and the present disclosure is not limited to this.

The present disclosure further provides a transcription unit comprising the nucleic acid or the pression cassette. The transcription unit refers to the DNA sequence beginning from the promoter and ending at the terminator. Regulatory fragments may also be comprised on either side of or between the promoter and terminator, and may include promoters, enhancers, transcription termination signals, polyadenylation sequences, origins of replication, nucleic acid restriction sites, and homologous recombination sites, such as enhancers of promoter and poly(A) signals, which are operably linked to the nucleic acid sequence. In the present disclosure, the promoter in the expression cassette is any one selected from the group consisting of a T7 promoter, a sCMV promoter, a Lac promoter, a tac promoter, an IPL promoter, an araB promoter, a Pzt-1 promoter and a trp promoter, and the terminator is any one selected from the group consisting of a T7 terminator, an rrnB T1 terminator, an ρ-independent terminator and a ρ-dependent terminator. For example, the expression cassette of the present disclosure has the following structural arrangement:
T7 promoter, nucleic acid fragment set forth in SEQ ID NO: 1, T7 terminator;
T7 promoter, nucleic acid fragment set forth in SEQ ID NO: 2, T7 terminator;
T7 promoter, nucleic acid fragment set forth in SEQ ID NO: 3, T7 terminator;
T7 promoter, nucleic acid fragment set forth in SEQ ID NO: 4, T7 terminator;
T7 promoter, nucleic acid fragment set forth in SEQ ID NO: 5, T7 terminator; or
T7 promoter, nucleic acid fragment set forth in SEQ ID NO: 6, T7 terminator.

Specifically, in some embodiments, the expression cassette of the present disclosure may further comprise an enhancer, an intron, a cofactor, a transcription element or another specific element. For example, the expression cassette of the present disclosure may comprise a promoter, an enhancer, a transcription element, any nucleic acid encoding a D-amino acid oxidase of the present disclosure, and a terminator. The present disclosure is not limited to this, and any expression cassette comprising the nucleic acid encoding a D-amino acid oxidase of the present disclosure is within the protection scope of the present disclosure.

The present disclosure provides a plasmid vector comprising a backbone vector and the nucleic acid encoding a D-amino acid oxidase of the present disclosure, or the expression cassette of the present disclosure. Further, the plasmid vector provided by the present disclosure further comprises a chaperone, and the chaperone is selected from the group consisting of PKJE7, PGro7, PTf16, PG-Tf2, PG-KJE8 and a combination thereof. The chaperone and the nucleic acid encoding a D-amino acid oxidase may or may not be present in the same expression cassette, and the present disclosure is not limited to this. In some specific embodiments, when the chaperone and the nucleic acid encoding a D-amino acid oxidase are present in the same expression cassette, they share the same promoter and terminator, and when they are not present in the same expression cassette, the chaperone and the nucleic acid encoding a D-amino acid oxidase each have a promoter and a terminator. In an embodiment of the present disclosure, the chaperone and the nucleic acid encoding a D-amino acid oxidase are not present in the same expression cassette, the promoter of the chaperone is a Pzt-1 promoter or an araB promoter, and its terminator is an rrnB T1 terminator. The backbone vector includes a pUC series vector, a pCAMBIA series vector, a pSC series vector and a pET series vector. It may be a shuttle vector, phage or viral vector, the present disclosure is not limited to this. It was found that the nucleic acid or expression cassette of the present disclosure constructed into a pET series vector showed a high expression by screening backbone vectors. In an embodiment of the present disclosure, the backbone vector is preferably pET-28a.

The plasmid vector of the present disclosure refers to a nucleic acid vector, which is a recombinant DNA molecule comprising a desired coding sequence and suitable nucleic acid sequences or elements necessary for the expression of an operably linked coding gene in a specific host organism. Nucleic acid sequences necessary for expression in bacteria include a promoter, a ribosome binding site and possibly other sequences. The plasmid vector of the present disclosure may be linear or circular and may be single stranded or double stranded, which is not limited by the present disclosure. The plasmid vector of the present disclosure comprises the expression cassette of D-amino acid oxidase aforementioned. Further, the plasmid vector of the present disclosure further comprises the expression cassette of the chaperone aforementioned.

When the chaperone and the expression cassette provided by the present disclosure are not present in the same plasmid vector, the present disclosure further provides a plasmid combination comprising a plasmid vector containing the nucleic acid encoding a D-amino acid oxidase of the present disclosure, and a plasmid vector containing a nucleic acid encoding the chaperone. The chaperone is selected from the group consisting of PKJE7, PGro7, PTf16, PG-Tf2, PG-KJE8 and a combination thereof. The chaperone is preferably PKJE7 (comprising a nucleic acid fragment set forth in SEQ ID NOs: 7 to 8), PGro7 (comprising a nucleic acid fragment set forth in SEQ ID NOs: 9 to 11) or PG-Tf2 (comprising a nucleic acid fragment set forth in SEQ ID NOs: 7 to 8 and 12). In an experiment of the present disclosure, it has been determined that the co-expression of the chaperone and the D-amino acid oxidase in the same plasmid vector offers a higher enzyme activity compared to the expression of the two in two separate vectors.

The present disclosure provides a host cell comprising at least one selected from the group consisting of I) and II):
I) a genome integrated with at least one nucleic acid encoding a D-amino acid oxidase of the present disclosure or the expression cassette of the present disclosure, and
II) the plasmid vector or the plasmid combination of the present disclosure by transfection or transformation.

In the present disclosure, the recombinant vector is transfected or transformed into a host. Transformation methods include chemical transformation and electro-transformation. Transfection methods include calcium phosphate co-precipitation, artificial liposome method, and viral transfection. The viral transfection methods include adenoviral transfection, adeno-associated viral transfection, lentiviral transfection, etc.

Further, the host of the present disclosure includes bacteria, fungi, viruses, and animals. The bacteria include Gram-positive and Gram-negative bacteria. The Gram-positive bacteria include but are not limited to Escherichia coli. The fungi include molds, yeasts, and macrofungi. The yeasts include baker's yeast, distiller's yeast, Pichia pastoris, and Candida yeast, etc. The viruses include but are not limited to adenoviruses, adeno-associated viruses, lentiviruses, and prions. The animals include humans, mice, rabbits, pigs, zebrafish, etc.

Specifically, in some embodiments, the host is Escherichia coli, and the Escherichia coli is any one strain selected from the group consisting of BL21 (DE3), BL21 Star (DE3), Shuffle T7, Tssetta, BL21 (DE3) PLysS, Rosetta-gami 2 and Origami 2. The Escherichia coli is preferably Shuffle T7 or BL21 (DE3).

The present disclosure provides use of the nucleic acid encoding a D-amino acid oxidase, the expression cassette, the plasmid vector, the plasmid combination or the host cell for producing a D-amino acid oxidase.

The present disclosure provides a method for producing a D-amino acid oxidase, comprising fermenting and culturing the host cell of the present disclosure. Specifically, the method comprises inoculating successfully constructed recombinant bacteria into liquid LB medium, culturing for 2 to 3 h, adding IPTG to a final concentration of 0.5 mM when the OD600 value indicating bacterial density reaches 0.8, culturing the bacteria at a low temperature for 16 h, centrifuging the resulting culture, resuspending and ultrasonicating for breaking the cells to obtain a crude enzyme solution.

The present disclosure provides a D-amino acid oxidase produced by the method of the present disclosure.

The present disclosure further provides use of the D-amino acid oxidase in the production of a α-keto acid.

The present disclosure further provides a method for producing a α-keto acid, comprising carrying out oxidization reaction on a substrate of D-amino acid or a racemic amino acid using the D-amino acid oxidase of the present disclosure. The D-amino acid oxidase is obtained by culturing and fermenting a host having a genome integrated with the nucleic acid or expression cassette containing the D-amino acid oxidase gene of the present disclosure, or by culturing and fermenting a host transfected or transformed with the plasmid vector or plasmid combination of the present disclosure.

Specifically, in the production of α-keto acid, the substrate has a concentration of 0.2 mol/L and the oxidation is carried out at a temperature of 30° C. for a time of 6 to 14 h. The reaction solution has a pH value of 8. Preferably, the oxidation is carried out at a temperature of 30° C. for a time of 6 h

In the present disclosure, 6 genes encoding wild-type D-amino acid oxidases from different sources are obtained by screening, which have a high expression level and the obtained D-amino acid oxidases have high enzyme activity. The enzyme activity and soluble expression of D-amino acid oxidase are further improved by optimizing genes for expression host and adding a chaperone.

The method of using DL-glufosinate as a substrate and performing kinetic resolution by using D-amino acid oxidase to obtain the corresponding keto acid and L-glufosinate has a simple process, mild reaction conditions, a low cost catalyst and an efficient and green process, and is an ideal solution for producing L-glufosinate, which can be widely used in the market.

### BRIEF DESCRIPTION OF DRAWINGS

FIG. 1 shows electrophoretograms of D-amino acid oxidase proteins in genetically engineered bacteria.

FIG. 2 shows the single plasmid profile for co-expression of DAAO27 and PGro7 chaperone.

FIG. 3 shows the electropherogram of the D-amino acid oxidase 27 (DAAO27) protein with the addition of the chaperone.

### DETAILED DESCRIPTION

The present disclosure provides a method for expressing a D-amino acid oxidase and use thereof. Those skilled in the art can refer to the contents of this disclosure and appropriately improve the process parameters to realize the present disclosure. It should be particularly noted that all similar substitutions and modifications are obvious to those skilled in the art, and they are deemed to be included in the present disclosure. The method and use of the present disclosure have been described through preferred embodiments, and it is apparent that relevant persons can make changes or appropriate modifications and combinations of the methods and use herein without departing from the content, spirit and scope of the present disclosure to implement and apply the present disclosure.

The materials and reagents used in the present disclosure are all commercially available and can be purchased from the market.

The recombinant Escherichia coli BL21 (DE3), BL21 Star (DE3), Shuffle T7, Tssetta, BL2 (DE3) PLysS, Rosetta-gami 2, and Origami 2 carrying the D-amino acid oxidase gene, and the used pET-28a(+) vector were purchased from TAKARA. The reagents used in the downstream catalytic process were as follows: D-alanine, D-aspartic acid, D-glutamic acid, D-serine, D-norvaline, D-phenylalanine, D-homophenylalanine, and DL-glufosinate purchased from Aladdin Bio-Chem Technology Co., LTD. Other commonly used reagents were purchased from Sinopharm Chemical Reagent Co., Ltd. The three-letter or single-letter code for representation of amino acids as specified by IUPAC (Eur. J. Biochem., 138: 9-37, 1984) is used in this application.

The concentration of the glufosinate was measured by high performance liquid chromatography (HPLC). Specifically, the chromatographic conditions were as follows. The chromatography column model was Pntulips® QS-C18 (5 μm, 4.6 mm×250 mm), the mobile phase was obtained by adding 0.9% of 10% aqueous tetrabutylammonium hydroxide solution to 50 mM diammonium hydrogen phosphate solution, adjusting the pH value to 3.6 with 50% phosphoric acid solution (mass fraction), and adding 8% acetonitrile, the detection wavelength was 205 nm, the flow rate was 0.8 mL/min, and the column temperature was 40° C.

The optical purity of the glufosinate was measured by high performance liquid chromatography using pre-column derivatization method. For derivatization reaction, 100 μL of the sample was added to 100 μL of derivatization reagent, mixed well and left at a holding temperature of 25° C. for 5 min. Chromatographic conditions used in the measurement were as follows. The chromatographic column was Pntulips® QS-C18, the detection wavelength was 338 nm, the column temperature was 30° C., the injection volume was 20 μL, the mobile phase was 50 mM aqueous sodium acetate:acetonitrile (9:0.5), and the flow rate was 1 mL/min.

7.62 g of sodium tetraborate decahydrate was weighed and added to deionized water, and the final volume was adjusted to 100 mL to obtain 0.2 M boric acid buffer for later use.

0.03 g of o-phthalaldehyde and 0.1 g of N-acetyl-L-cysteine were weighed, added with 400 μL of anhydrous ethanol and 4 mL of boric acid buffer, and completely dissolved by ultrasonic treatment to obtain the derivatization reagent, which was prepared just before use.

DAAO1 derived from Rhodotorula graminis WP1 has the nucleic acid sequence of:

(SEQ ID NO: 1)
ATGGCTATAGATAAAAGGGTAGTTGTCCTAGGCACGGGTGTTGTTGGTCT
GTCTTGCGGTCTGGTCTTGTCTCGTCAGGGCTATCGTGTTCACTTTATCG
CTCGTGACTTGCCAGAGGACAGTACCAGCCAGGGTTTCGCGTCGCCGTGG
GCAGGCGCGAACTGGACCCCGTTTTATAGCCGTGACGAGGGCCCAAGACA
AGCAAAGTGGGAAGAAGCCACCTTCGCACGTTGGGTTTCTCTCGTGCCGA
GCGGTCTGGCTATGTGGTTGAATGATACGCGTAGGTACGCGGATACCGAT
GCAGGTCTTCTGGGTCATTGGTATCGCGACACCGTCCGTAATTATCGTGA
ACTGCCGCCTTCTGAGCTGCCGAAGGGTGTTGCGGCGGGTGCGGCATACG
ACACTCTGTCTGTGAACGCTCCGCTGTATTGCCAGGCGCTGGCGCGTGAG
CTGCAGACCTTGGGTGCTACCTTCGAGCGCCGTAGCGTTTCAAGCATCGA
GCAGGTATTTGAGGGTCAGGACGACATCGCCCTGGTGGTGAACGCCACGG
GTCTGGGTGCGAAATCCATCGCGGGGATCGAAGATTCCGCCTGCCATCCG
GTTCGTGGTCAAACCGTGTTGGTTAAAAGCGGCTGCAAACGTTGTACCAT
GGATAGCAGCAATCCGGAAGCGCCGGCGTACATTATTCCGCGTCCGGGTG
GAGAAGTTATTTGCGGTGGCACCTACCTGGTCGATGACTGGGATTTGAGC
CCGTCCGCGAGCACGGCGCAACGTATTCTGACCCAATGTCTGGCGTTAGA
TCCGTCGATCAGCACTGACGGCACTCTGGATGGCATCCACATTCTGCGTC
ATAACGTGGGCTTGCGCCCAGCGCGTACCGGCGGTCCGCGCGTCGAGGTG
GGCAAACTGACCCTTCCGCTCGTTCGCTCCACGGAACCGGGTACTGCGCT
TGCTCTGGGCACCGCGCGCCCTGCGCCGGCAGGCGCGAGCAGCGAGGCAG
TTGGTGCCCCGTCGGAGGCTGTGAAGCGCGAGGTGACCCTGGTTCACGCA
TACGGCTTCAGCTCTGCTGGTTACCAGCAAAGCTGGGGCGTGGCCCAAGA
CGTGCTCGGCCTGGTGGAAGGCGAAATTGGTCCGCCGCGCGCATGGTGGA
CCCAGAGAGGCAAGCTATAA.

DAAO2 derived from Vanrija humicola has the nucleic acid sequence of:

(SEQ ID NO: 2)
ATGCCACCTTCAGATCCCATAATTGTACTAGGCGCGGGCGTCATCGGCCT
GACGACCGCGGTTCGTTTACTGGAGGCACACCTAGGCGCGAACGTGCACA
TCCTGGCGGATCATTGGCCGTCCGATGCCCTGGACGCACAATATGCGTCC
ACCATTGCAGGCGCTCACCACCTCTCGTTCGCCGATGACGGTGATGCGCG
CCAACGTCGTTGGGATATGCGCACATTTGATGTTCTGTACGACGAATGGA
AGGCTGTTGGCGAGCGTACTGGTCTTATGGCATTGACGCAGACGGAAATG
TGGGAGGGTGCTACGAGCCATTTAGCGGTTTATGAAGGCAATCCGGACTT
CCGCGTACTGGACCCGCGTACCGCACCGTGCAGCAACATTACCCATATGG
TTAGCTTTACCAGTCTGACCATTGCTCCGACCGTTTACCTGGCCGCTCTG
GAGGCTCGTGTGCGCGACCTGGGTGCCAAACTCCACCGTGCCCACGTGCC
GTCTCTGGGCGCGCTTCGCACCGACCCAGCGCTGCTGGCGTTGTACACCC
GTCCGCCTGCAGCGGTTTTTGTTTGTGCGGGTCTGGGTGCGCGCCATCTG
GTTCCGGCTCCGGAAGCGGCAGCGTTGTTTCCGACCCGTGGTCAGGTCGT
CGTTGTCCGTGCGCCTTGGATGCGTGCAGGCTTCACTAGACAAGTTGGTA
GCCTGGGCGGCGGCGAGGGTGGCACCCGTACGTACATTATCCCGCGTTGT
AATGGTGAGGTTGTGTTGGGCGGTACCATGGAACAGGGTGACTGGACTCC
GTACCCGAGAGACGAGACCGTTACCGACATCCTGACCCGTGCACTGCAAA
TTTGCCCGGATATCGCGCCACCGTATGCGCGGTCTTGGCCGAAAGATGAT
CAGGTGGCTGCGCTGAGAAGCATCGTGGTGCGTGACGCGGTGGGTTTCCG
CCCGAGCCGTGCTGGAGGCGCGCGTGTCGCACTGGCGAGCGCTGCGGGTA
TGCGAGTGGTGTACAACTATGGTCACGGTGGCGCCGGTTGGCAGTCCTGC
TGGGGGTGCGCAGAAGATGCCGTGGCCCTGTGGGCAGGTGGTGCGGGCGG
CGCTCGCTTGTAA.

DAAO3 derived from Bos taurus has the nucleic acid sequence of:

(SEQ ID NO: 3)
ATGGATACAGTTAGGATAGCTGTAGTCGGAGCGGGTGTAATGGGTTTGTC
AACCGCAGTTTGTATTTCCAAAATGGTACCGGGGTGCTCCATCACCGTTA
TCTCCGACAAATTCACGCCGGAAACAACCAGCGACGTGGCCGCAGGCATG
CTTATCCCACCGACGTACCCGGATACCCCGATTCAGAAGCAGAAGCAGTG
GTTCAAAGAAACCTTTGACCACCTGTTTGCGATTGTGAACAGCGCTGAGG
CCGAGGACGCTGGCGTCATCCTGGTTTCCGGCTGGCAGATTTTCCAAAGC
ATTCCGACGGAGGAAGTTCCTTATTGGGCGGATGTGGTCCTGGGTTTTCG
TAAAATGACCAAAGATGAATTGAAGAAATTCCCACAGCATGTTTTTGGTC
ATGCGTTTACGACCCTGAAGTGCGAAGGTCCGGCGTACCTGCCGTGGCTG
CAAAAGCGCGTGAAAGGAAACGGTGGTCTAATCCTGACCCGTCGTATTGA
GGACCTGTGGGAACTGCATCCGAGCTTCGACATCGTGGTCAACTGCTCTG
GCTTGGGCAGCCGTCAACTGGCGGGCGACTCGAAGATCTTCCCGGTTAGA
GGCCAGGTTCTGAAAGTGCAAGCGCCCTGGGTTAAACACTTTATCCGTGA
TTCTAGCGGCTTGACCTACATCTATCCGGGTGTGAGCAACGTGACCCTGG
GTGGCACCCGCCAAAAGGGTGATTGGAATCTGTCGCCGGACGCCGAAATC
AGCAAGGAGATCCTCTCTCGTTGTTGCGCGCTCGAGCCGAGCCTGCGTGG
TGCATATGATCTGCGTGAAAAGGTGGGTCTGCGTCCGACTCGCCCATCTG
TTCGTCTGGAGAAAGAATTGCTGGCGCAGGATTCGAGACGTTTACCGGTC
GTGCACCACTACGGCCACGGCAGCGGTGGTATTGCAATGCATTGGGGTAC
TGCCTTGGAGGCGACCCGCCTTGTTAATGAGTGCGTTCAAGTTCTGCGCA
CCCCGGCTCCGAAAAGCAAGCTGTAA.

DAAO4 derived from Caenorhabditis elegans has the nucleic acid sequence of:

(SEQ ID NO: 4)
ATGCCCAAAATAGCAGTACTAGGAGCTGGGATCAACGGCATCGCCAGCGC
GCTTGCCATTCAAGAACGTCTTCCGAACTGCGAAGTTACCATCATCGCGG
AGAAATTCAGCCCGAACACCACCTCCGATGTGGCGGCAGGCCTGATTGAA
CCATTTCTGTGTGATGACGACGTGGATCGTATCATCAATTGGACCTCTGC
CACCATCAGCCGCATCCACGAATACCAGGCGGATGGTAATCCGGGTGCAG
AAGAGCAGAGCGGCTACTGGCTCCAAAGCGTTAAAAGCGAACCGAAGTGG
TTGAAGCTGATGAAAAACGTGCACATTCTAACTGACGCGGAGATGAAACA
AGTAGCTCGCCGTCCTGAACATAAATTCGGCATTTTTTATACCACGTGGT
ACCTGGAACCGACTCCGTATATTAAATGGTGTACCGACAAGTTCCTGAAA
AACGGTGGCAAATTCAAGAAACAGAAAATTGAGAACATCGATGACGTCGC
GCGCAGCTATGATGTGACCGTTAATTGCACCGGTCTGGGCAGCCGCGCTC
TGATTGGTGATAAAGAGGTTTACCCGACGCGTGGTCAAATTTTGAAGGTG
AGCTGCCCGCGTGTTAAGCACTTTTTCATCGACGACAAGTATTACGCTTT
ACTGAATGATAGCACCATTACCTTGGGCGGCACGTTTGAAGCACATCAGT
GGGACCTGACGATCAACTCCGAGCTGTCACAGAAAATTCTCAAGGAGAAT
ATCCACAATATCCCGTCGCTGCGTACCGCGCAGATTTTGTCTTCCCACGT
GGACATGCGTCCGAGCAGAGGCACGGTTCGTTTGCAAGCGGAGCTGGGGC
GTTCGCTGGTTCACAACTATGGTCATGGTGGTTCTGGTATTACCCTGCAT
TGGGGTTGCGCGTTGGAGTGCGCTGAGATCGTGGAAAACGTCCTGAAGAT
GAAGAAGTCCAAACTGTAA.

DAAO5 derived from Caenorhabditis elegans has the nucleic acid sequence of:

(SEQ ID NO: 5)
ATGACACCCAAAATTGCTATAATAGGAGAAGGCGTCATCGGCTGCTCCAC
CGCACTGCAGGTTGCGCAGGCGGTGCCTGATGCACGCGTGACGGTTTTGT
CTGACCGTCCGTTTGAACAGACCTGTTCTTTTGGACCGGCTGGTCTGTTC
CGCATTGATGATATTGCGAACCGTGAGTTCGGCAAGAGCACCTTCGACTG
GTTCGCCCACCTGCATCGTACCGAAAAGGGTGACAAAACCGGCGTGAAGT
TGCTGAGCGGTCATATTCAAAGCGATTCCAAAGAACGTCTGGAGCAGCAA
CAAAAAGCGTATGGTGACATTGTGTACAATTTTCGTTTCCTGGAGAAGCG
CGAGATCCTCGACCTGTTCCCGAATCCGAGCGAGCACTGCATCCACTACA
CTGCGTTTGCGAGCGAAGGCAACAAATACGTTCCGTATCTGAAATTCCAG
TGTCAGGCACGTGGCGTGGAGTTTCTGCACAGAAAGGTGCGTGATTTGGA
GGAACTGGCGAACGAAGGTTATGATGTGATCGTGAACTGCGCAGGCTTAT
CCGGCGGTACGCTGGCGGGCGACGACGACAGCGTCTACCCGATTCGTGGT
GTTGTCCTGGACGTTGAAGCCCATTGGCACAAACACTTCAACTATAAAGA
TTTTATCACCTTCACCATTCCGAAAGAGAACTCTGTAGTTATTGGCAGCG
TGAAACAAGAAAACCGCTGGGATTTGGAGATCACCGATGTTGACCGCAAG
GACATCTTGGAGCGCTATGTGGCCCTGCATCCGGCTATGCGTGAACCAAA
AATCCTGGGTGAATGGTCAGGTCTGCGTCCGGCTCGTAAGACGATCCGTA
TTGAAAAAGTTGAGAAGAAGTCGGAAAAGTCGGGCAAAAAGTACACTGTT
GTCCACCACTACGGCCATGGTGGTAATGGTTTTACCCTCGGTTGGGGTAC
AGCCGTGGAGGCTACCAAATTAGTTAAGTCCGCGCTGAATAGCAGCAAGC
TT.

DAAO27 derived from Neurospora crassa OR74A has the nucleic acid sequence of:

(SEQ ID NO: 6)
ATGCATCTGCGTTTTCCGACCACCACCACCTATAGTCTGCGCCCGAGCAG
CACCCGCACCAGCCCTCCTCCTCTGATTGTTAGCTTTGCCCCGCGCCATA
GCATTGTGAGTTATACCACCAGTCCGCGCCGCCTGTTTAGTATTATTATG
AGCGAACCGAAAGGCCATGTGGTTGTTATTGGCGCAGGCGTGATTGGTCT
GAGCAGCGCCCTGGCACTGCTGGAAGCAAATTATGCAACCACCATTCTGG
CCAAAGATCTGCCGGCACCGTTTGAAAGTATTGATCCGCGTAGCCAGATT
AATTTTACCAGCCCGTGGGGTGGTGCACATAATCGTTGGGTGCCGCCGTT
TCCGCCGAGTAGTAGTAGCAGCAGTACCCCGCTGACCCCGACCCAGCAGG
CAGAACATGCACTGCGTATTCGTGAACATGCATTTAGCCTGAGCACCTTT
CATCGTATGCAGCTGTTTCAGAGCCAGGGTCAGGATCAGCAGGCAGGCAT
TACCTTTCTGAAAGGCATTGAATATCTGGAAAGCCCGGGCCCGGAATATA
CCAGCCTGACCCCGCAGCGTGCCAGTCAGGAACTGGGCCTGCCGGGTCAT
TTTCGTGTGCTGGAAACCCATGAATTTCCGGATGATAAAGTTCAGTGGGG
TTGCGAATATGATACCTGGTGCGTGAATCCGATGCAGTATTGCCTGTTTC
TGCTGGGTCAGATTATTGCACGCGGTGGTAAAGTGTTTAAACGTGATGTG
CGTAGCGTTGCAGAAGTGTTTCAGCTGTTTAGTGATAGCGTTCAGGAATT
TGGTGCCACCATTCCGCCGGCCGATGCAGTGGTGAATGCAACCGGTATTG
GCCTGGGCGATGATGAAATGGTGTTTCCGACCCGTGGTCAGACCTGCCTG
GTGCAGGAACCGTGCGATGCAACCGTGACCCGCCAGAATGCCGATGGTAC
CTGGACCTTTTGTGTGCCGCGTGGCTTTAAAGCAGGTACCATTATTGGCG
GCACCAAAGAACCGGATAATTGGGATCCGAAACCGGATCCGGAAGTTCGT
GAACGTCTGCTGCGTGCCTTTGAAGGTACCTATCCGCGTATTCTGGCCGA
TGGCAAAACCCGCCTGACCCCGGTGCGCGATATTGTGGGTCGTCGTCCGA
CCCGCAAAGGTGGCCTGCGTCTGGAAGGCGAAGTGGTTGATGGTGCCGGT
TTTGTGATGCATGCCTATGGCCTGGGCGGTCGTGGTTATGAACTGAGTTG
GGGTGTTGCCGAAGGTGTTATTGAAGGCATTCAGGGTCATCTGGAAAGCG
AAAAAGGCAGTCGTCTGTAA.

PGro7 comprises two chaperone proteins of groES and groEL:

groES:
(SEQ ID NO: 7)
atgaatattcgtccattgcatgatcgcgtgatcgtcaagcgtaaagaagt
tgaaactaaatctgctggcggcatcgttctgaccggctctgcagcggcta
aatccacccgcggcgaagtgctggctgtcggcaatggccgtatccttgaa
aatggcgaagtgaagccgctggatgtgaaagttggcgacatcgttatttt
caacgatggctacggtgtgaaatctgagaagatcgacaatgaagaagtgt
tgatcatgtccgaaagcgacattctggcaattgttgaagcgtaa
groEL:
(SEQ ID NO: 8)
atggcagctaaagacgtaaaattcggtaacgacgctcgtgtgaaaatgct
gcgcggcgtaaacgtactggcagatgcagtgaaagttaccctcggtccaa
aaggccgtaacgtagttctggataaatctttcggtgcaccgaccatcacc
aaagatggtgtttccgttgctcgtgaaatcgaactggaagacaagttcga
aaatatgggtgcgcagatggtgaaagaagttgcctctaaagcaaacgacg
ctgcaggcgacggtaccaccactgcaaccgtactggctcaggctatcatc
actgaaggtctgaaagctgttgctgcgggcatgaacccgatggacctgaa
acgtggtatcgacaaagcggttaccgctgcagttgaagaactgaaagcgc
tgtccgtaccatgctctgactctaaagcgattgctcaggttggtaccatc
tccgctaactccgacgaaaccgtaggtaaactgatcgctgaagcgatgga
caaagtcggtaaagaaggcgttatcaccgttgaagacggtaccggtctgc
aggacgaactggacgtggttgaaggtatgcagttcgaccgtggctacctg
tctccttacttcatcaacaagccggaaactggcgcagtagaactggaaag
cccgttcatcctgctggctgacaagaaaatctccaacatccgcgaaatgc
tgccggttctggaagctgttgccaaagcaggcaaaccgctgctgatcatc
gctgaagatgtagaaggcgaagcgctggcaactctggttgttaacaccat
gcgtggcatcgtgaaagtcgctgcggttaaagcaccgggcttcggcgatc
gtcgtaaagctatgctgcaggatatcgcaaccctgactggcggtaccgtg
atctctgaagagatcggtatggagctggaaaaagcaaccctggaagacct
gggtcaggctaaacgtgttgtgatcaacaaagacaccaccactatcatcg
atggcgtgggtgaagaagctgcaatccagggccgtgttgctcagatccgt
cagcagattgaagaagcaacttctgactacgaccgtgaaaaactgcagga
acgcgtagcgaaactggcaggcggcgttgcagttatcaaagtgggtgctg
ctaccgaagttgaaatgaaagagaaaaaagcacgcgttgaagatgccctg
cacgcgacccgtgctgcggtagaagaaggcgtggttgctggtggtggtgt
tgcgctgatccgcgtagcgtctaaactggctgacctgcgtggtcagaacg
aagaccagaacgtgggtatcaaagttgcactgcgtgcaatggaagctccg
ctgcgtcagatcgtattgaactgcggcgaagaaccgtctgttgttgctaa
caccgttaaaggcggcgacggcaactacggttacaacgcagcaaccgaag
aatacggcaacatgatcgacatgggtatcctggatccaaccaaagtaact
cgttctgctctgcagtacgcagcttctgtggctggcctgatgatcaccac
cgaatgcatggttaccgacctgccgaaaaacgatgcagctgacttaggcg
ctgctggcggtatgggcggcatgggtggcatgggcggcatgatgtaa

PKJE7 comprises three chaperone proteins of dnaK, dnaJ and grpE:

dnaK:
(SEQ ID NO: 9)
ATGGGTAAAATAATTGGTATCGACCTGGGTACTACCAACTCTTGTGTAGCGAT
TATGGATGGCACCACTCCTCGCGTGCTGGAGAACGCCGAAGGCGATCGCACCACGCC
TTCTATCATTGCCTATACCCAGGATGGTGAAACTCTAGTTGGTCAGCCGGCTAAACGTC
AGGCAGTGACGAACCCGCAAAACACTCTGTTTGCGATTAAACGCCTGATTGGTCGCC
GCTTCCAGGACGAAGAAGTACAGCGTGATGTTTCCATCATGCCGTTCAAAATTATTGC
TGCTGATAACGGCGACGCATGGGTCGAAGTTAAAGGCCAGAAAATGGCACCGCCGCA
GATTTCTGCTGAAGTGCTGAAAAAAATGAAGAAAACCGCTGAAGATTACCTGGGTGA
ACCGGTAACTGAAGCTGTTATCACCGTACCGGCATACTTTAACGATGCTCAGCGTCAG
GCAACCAAAGACGCAGGCCGTATCGCTGGTCTGGAAGTAAAACGTATCATCAACGAA
CCGACCGCAGCTGCGCTGGCTTACGGTCTGGACAAAGGCACTGGCAACCGTACTATC
GCGGTTTATGACCTGGGTGGTGGTACTTTCGATATTTCTATTATCGAAATCGACGAAGT
TGACGGCGAAAAAACCTTCGAAGTTCTGGCAACCAACGGTGATACCCACCTGGGGGG
TGAAGACTTCGACAGCCGTCTGATCAACTATCTGGTTGAAGAATTCAAGAAAGATCA
GGGCATTGACCTGCGCAACGATCCGCTGGCAATGCAGCGCCTGAAAGAAGCGGCAGA
AAAAGCGAAAATCGAACTGTCTTCCGCTCAGCAGACCGACGTTAACCTGCCATACATC
ACTGCAGACGCGACCGGTCCGAAACACATGAACATCAAAGTGACTCGTGCGAAACTG
GAAAGCCTGGTTGAAGATCTGGTAAACCGTTCCATTGAGCCGCTGAAAGTTGCACTG
CAGGACGCTGGCCTGTCCGTATCTGATATCGACGACGTTATCCTCGTTGGTGGTCAGA
CTCGTATGCCAATGGTTCAGAAGAAAGTTGCTGAGTTCTTTGGTAAAGAGCCGCGTAA
AGACGTTAACCCGGACGAAGCTGTAGCAATCGGTGCTGCTGTTCAGGGTGGTGTTCT
GACTGGTGACGTAAAAGACGTACTGCTGCTGGACGTTACCCCGCTGTCTCTGGGTATC
GAAACCATGGGCGGTGTGATGACGACGCTGATCGCGAAAAACACCACTATCCCGACC
AAGCACAGCCAGGTGTTCTCTACCGCTGAAGACAACCAGTCTGCGGTAACCATCCAT
GTGCTGCAGGGTGAACGTAAACGTGCGGCTGATAACAAATCTCTGGGTCAGTTCAAC
CTAGATGGTATCAACCCGGCACCGCGCGGCATGCCGCAGATCGAAGTTACCTTCGATA
TCGATGCTGACGGTATCCTGCACGTTTCCGCGAAAGATAAAAACAGCGGTAAAGAGC
AGAAGATCACCATCAAGGCTTCTTCTGGTCTGAACGAAGATGAAATCCAGAAAATGG
TACGCGACGCAGAAGCTAACGCCGAAGCTGACCGTAAGTTTGAAGAGCTGGTACAGA
CTCGCAACCAGGGCGACCATCTGCTGCACAGCACCCGTAAGCAGGTTGAAGAAGCA
GGCGACAAACTGCCGGCTGACGACAAAACTGCTATCGAGTCTGCGCTGACTGCACTG
GAAACTGCTCTGAAAGGTGAAGACAAAGCCGCTATCGAAGCGAAAATGCAGGAACT
GGCACAGGTTTCCCAGAAACTGATGGAAATCGCCCAGCAGCAACATGCCCAGCAGCA
GACTGCCGGTGCTGATGCTTCTGCAAACAACGCGAAAGATGACGATGTTGTCGACGC
TGAATTTGAAGAAGTCAAAGACAAAAAATAA
dnaJ:
(SEQ ID NO: 10)
ATGGCTAAGCAAGATTATTACGAGATTTTAGGCGTTTCCAAAACAGCGGAAG
AGCGTGAAATCAGAAAGGCCTACAAACGCCTGGCCATGAAATACCACCCGGACCGTA
ACCAGGGTGACAAAGAGGCCGAGGCGAAATTTAAAGAGATCAAGGAAGCTTATGAA
GTTCTGACCGACTCGCAAAAACGTGCGGCATACGATCAGTATGGTCATGCTGCGTTTG
AGCAAGGTGGCATGGGCGGCGGCGGTTCTGGCGGCGGCGCAGACTTCAGCGATATTT
TTGGTGACGTTTTCGGCGATATTTTTGGCGGCGGACGTGGTCGTCAACGTGCGGCGCG
CGGTGCTGATTTACGCTATAACATGGAGCTCACCCTCGAAGAAGCTGTACGTGGCGTG
ACCAAAGAGATCCGCATTCCGACTCTGGAAGAGTGTGACGTTTGCCACGGTAGCGGT
GCAAAACCAGGTACACAGCCGCAGACTTGTCCGACCTGTCATGGTTCTGGTCAGGTG
CAGATGCGCCAGGGATTCTTCGCTGTACAGCAGACCTGTCCACACTGTCAGGGCCGC
GGTACGCTGATCAAAGATCCGTGCAACAAATGTCATGGTCATGGTCGTGTTGAGCGCA
GCAAAACGCTGTCCGTTAAAATCCCGGCAGGGGTGGACACTGGAGACCGCATCCGTC
TTGCGGGCGAAGGTGAAGCGGGCGAGCATGGCGCACCGGCAGGCGATCTGTACGTTC
AGGTTCAGGTTAAACAGCACCCGATTTTCGAGCGTGAAGGCAACAACCTGTATTGCG
AAGTCCCGATCAACTTCGCTATGGCGGCGCTGGGTGGCGAAATCGAAGTACCGACCC
TTGATGGTCGCGTCAAACTGAAAGTGCCTGGCGAAACCCAGACCGGTAAGCTATTCC
GTATGCGCGGTAAAGGCGTCAAGTCTGTCCGCGGTGGCGCGCAGGGTGATTTGCTGT
GCCGCGTTGTCGTCGAAACACCGGTAGGCCTGAACGAAAGGCAGAAACAGCTGCTG
CAAGAGCTGCAAGAAAGCTTCGGTGGCCCAACCGGCGAGCACAACAGCCCGCGCTC
AAAGAGCTTCTTTGATGGTGTGAAGAAGTTTTTTGACGACCTGACCCGCTAA
grpE:
(SEQ ID NO: 11)
ATGAGTAGTAAAGAACAGAAAACGCCTGAGGGGCAAGCCCCGGAAGAAATT
ATCATGGATCAGCACGAAGAGATTGAGGCAGTTGAGCCAGAAGCTTCTGCTGAGCAG
GTGGATCCGCGCGATGAAAAAGTTGCGAATCTCGAAGCTCAGCTGGCTGAAGCCCAG
ACCCGTGAACGTGACGGCATTTTGCGTGTAAAAGCCGAAATGGAAAACCTGCGTCGT
CGTACTGAACTGGATATTGAAAAAGCCCACAAATTCGCGCTGGAGAAATTCATCAACG
AATTGCTGCCGGTGATTGATAGCCTGGATCGTGCGCTGGAAGTGGCTGATAAAGCTAA
CCCGGATATGTCTGCGATGGTTGAAGGCATTGAGCTGACGCTGAAGTCGATGCTGGAT
GTTGTGCGTAAGTTTGGCGTTGAAGTGATCGCCGAAACTAACGTCCCACTGGACCCG
AATGTGCATCAGGCCATCGCAATGGTGGAATCTGATGACGTTGCGCCAGGTAACGTAC
TGGGCATTATGCAGAAGGGTTATACGCTGAATGGTCGTACGATTCGTGCGGCGATGGTT
ACTGTAGCGAAAGCAAAAGCTTAA

PG-Tf2 comprises three chaperone proteins of groES, groEL and tig, and the sequences of groES and groEL are the same as disclosed above.

tig:
(SEQ ID NO. 12)
atgcaagtttcagttgaaaccactcaaggccttggccgccgtgtaacgat
tactatcgctgctgacagcatcgagaccgctgttaaaagcgagctggtca
acgttgcgaaaaaagtacgtattgacggcttccgcaaaggcaaagtgcca
atgaatatcgttgctcagcgttatggcgcgtctgtacgccaggacgttct
gggtgacctgatgagccgtaacttcattgacgccatcattaaagaaaaaa
tcaatccggctggegcaccgacttatgttccgggcgaatacaagctgggt
gaagacttcacttactctgtagagtttgaagtttatccggaagttgaact
gcagggtctggaagcgatcgaagttgaaaaaccgatcgttgaagtgaccg
acgctgacgttgacggcatgctggatactctgcgtaaacagcaggcgacc
tggaaagaaaaagacggcgctgttgaagcagaagaccgcgtaaccatcga
cttcaccggttctgtagacggcgaagagttcgaaggcggtaaagcgtctg
atttcgtactggcgatgggccagggtcgtatgatcccgggctttgaagac
ggtatcaaaggccacaaagctggcgaagagttcaccatcgacgtgacctt
cccggaagaataccacgcagaaaacctgaaaggtaaagcagcgaaattcg
ctatcaacctgaagaaagttgaagagcgtgaactgccggaactgactgca
gaattcatcaaacgtttcggcgttgaagatggttccgtagaaggtctgcg
cgctgaagtgcgtaaaaacatggagcgcgagctgaagagcgccatccgta
accgcgttaagtctcaggcgatcgaaggtctggtaaaagctaacgacatc
gacgtaccggctgcgctgatcgacagcgaaatcgacgttctgcgtcgcca
ggctgcacagcgtttcggtggcaacgaaaaacaagctctggaactgccgc
gcgaactgttcgaagaacaggctaaacgccgcgtagttgttggcctgctg
ctgggcgaagttatccgcaccaacgagctgaaagctgacgaagagcgcgt
gaaaggcctgatcgaagagatggcttctgcgtacgaagatccgaaagaag
ttatcgagttctacagcaaaaacaaagaactgatggacaacatgcgcaat
gttgctctggaagaacaggctgttgaagctgtactggcgaaagcgaaagt
gactgaaaaagaaaccactttcaacgagctgatgaaccagcaggcgtaa

PTf16 comprises one tig chaperone with the same sequence as disclosed above.

PG-KJE8 comprises five chaperones of dnaK, dnaJ, grpE, groES and groEL, with the same sequences as disclosed above.

The present disclosure is further illustrated below in conjunction with examples.

### Example 1 Construction of Genetically Engineered Bacterium Expressing Wild-Type D-Amino Acid Oxidase and Vitality Determination

### 1. Obtaining D-Amino Acid Oxidase Genes

The source of D-amino acid oxidase and gene sequence related information were collected through literature research and multiple sequence comparison, gene sequences or amino acid sequences of D-amino acid oxidases of different sources were searched in the gene database (https://www.ncbi.nlm.nih.gov/genome/), and the whole gene synthesis was performed. The specific detail of D-amino acid oxidase obtained is shown in Table 1.

TABLE 1
Details of D-amino acid oxidase

| NCBI accession number | Original gene source | Numbering |
|---|---|---|
| XP_018272978.1 | Rhodotorula graminis WP1 | DAAO1 |
| BAD13387.1 | Vanrija humicola | DAAO2 |
| CAA64622.1 | Bos taurus | DAAO3 |
| BAF34313.1 | Caenorhabditis elegans | DAAO4 |
| BAF34314.1 | Caenorhabditis elegans | DAAO5 |
| TNY23393.1 | Rhodotorula diobovata | DAAO6 |
| ALM22238.1 | Sporobolomyces roseus | DAAO7 |
| ORY54161.1 | Leucosporidium creatinivorum | DAAO8 |
| QDC12421.1 | Homo saapiens | DAAO9 |
| CED84830.1 | Phaffia rhodozyma | DAAO10 |
| BAC28253.1 | Mus musculus (Mouse) | DAAO11 |
| BAF32940.1 | Mus musculus (Mouse) | DAAO12 |
| BAF47961.1 | Sus scrofa (Pig) | DAAO13 |
| BAF34315.1 | Caenorhabditis elegans | DAAO14 |
| BAF34316.1 | Caenorhabditis elegans | DAAO15 |
| ALM22233.1 | Rhodotorula toruloides | DAAO16 |
| CAJ87425.1 | synthetic construct | DAAO17 |
| NP_001370668.1 | Caenorhabditis elegans | DAAO18 |
| UnProt P80324 | Rhodotorula toruloides | DAAO19 |
| CAA90322.1 | Trigonopsis variabilis | DAAO20 |
| BAB12222.1 | [Candida] boidinii | DAAO21 |
| BAA00692.1 | Fusarium solani | DAAO22 |
| EYB24484.1 | Fusarium graminearum | DAAO23 |
| OBS19408.1 | Fusarium poae | DAAO24 |
| XP_016270247.1 | Rhodotorula toruloides NP11 | DAAO25 |
| XP_001401161.2 | Aspergillus niger CBS 513.88 | DAAO26 |
| XP_964990 | Neurospora crassa OR74A | DAAO27 |

### 2. Construction of Bacteria Strain Expressing D-Amino Acid Oxidase

The codon optimization of D-amino acid oxidase gene sequence and the whole gene synthesis were carried out by a gene synthesis company. The synthesized gene was constructed into the pET-28a(+) plasmid vector via enzyme cleavage sites NheI and NotI. The constructed plasmid was introduced into the expression host of E. coli BL21 (DE3) strain to obtain the genetically engineered strain E. coli BL21 (DE3)/pET-28a(+)-DAAOx.

### 3. Expression of Recombinant D-Amino Acid Oxidase

The successfully constructed engineered bacteria were inoculated into liquid LB medium and cultured in a shaking incubator with a speed of 200 rpm at 37° C. for 2-3 h. When the OD600 value indicating the bacterial density reached 0.8, the bacteria were cooled to a temperature of 18° C. and added with IPTG to achieve a final concentration of 0.5 mM. Then, the triangular flask was transferred to a 18° C. shaking incubator with a speed of 200 rpm and the bacteria were cultured at 200 rpm for 16 h. After which, the culture was centrifuged at 4000 rpm for 30 min, the supernatant was discarded and the cells were collected. These cells were then resuspended in 100 mM phosphate buffer (pH 8.0), and were broken by ultrasonic treatment to obtain the bacterial suspension as the crude enzyme solution.

### 4. Enzyme Activity Assay of Recombinant D-Amino Acid Oxidase

The enzyme activity of recombinant D-amino acid oxidase was tested using DL-glufosinate as the substrate, and the assay system was as follows. The total reaction system was 2 mL, consisting of 1 mL of 200 mM DL-glufosinate solution and 1 mL of crude enzyme solution, and the above substances were formulated with 100 mM, pH 8.0 phosphate buffer. The assay system was shaken at 30° C. for 6 h to perform reaction, and 200 μL of 4 M HCl solution was added to terminate the reaction. The reaction mixture was centrifuged at 4000 rpm for 10 min to remove cells and enzyme proteins. High performance liquid chromatography was adopted to determine the PPO produced in the reaction system. One Unit (1 U) of enzyme activity is defined as that conversing substrate to produce 1 μmol product at 30° C. in 1 min. The enzyme activity of all recombinant D-amino acid oxidases was assayed and the results are shown in Table 2. As can be seen from Table 2, the enzyme activity was significantly increased by codon optimization of exogenous genes for Escherichia coli expression, wherein the optimized D-amino acid oxidases DAAO1, DAAO2, DAAO3, DAAO4, DAAO5 and DAAO27 were detected to have enzyme activity on the substrate DL-glufosinate, which are derived from Rhodotorula graminis WP1, Vanrija humicola, Bos Taurus, Caenorhabditis elegans and Neurospora crassa OR74A.

TABLE 2
Enzyme activity assay of D-amino acid oxidases (DAAO)

| | | Enzyme activity | Enzyme activity |
|---|---|---|---|
| | | (U/L), | (U/L), |
| DAAO | NCBI accession | without codon | with codon |
| number | number | optimization | optimization |
| DAAO1 | XP_018272978.1 | 0.89 ± 0.04 | 1.14 ± 0.21 |
| DAAO2 | BAD13387.1 | 0.34 ± 0.12 | 1.37 ± 0.33 |
| DAAO3 | CAA64622.1 | 0.00 ± 0.00 | 1.69 ± 0.29 |
| DAAO4 | BAF34313.1 | 0.87 ± 0.07 | 0.98 ± 0.19 |
| DAAO5 | BAF34314.1 | 1.55 ± 0.17 | 2.93 ± 0.26 |
| DAAO6 | TNY23393.1 | 0.00 ± 0.00 | 0.00 ± 0.00 |
| DAAO7 | ALM22238.1 | 0.00 ± 0.00 | 0.00 ± 0.00 |
| DAAO8 | ORY54161.1 | 0.00 ± 0.00 | 0.00 ± 0.00 |
| DAAO9 | QDC12421.1 | 0.00 ± 0.00 | 0.00 ± 0.00 |
| DAAO10 | CED84830.1 | 0.00 ± 0.00 | 0.00 ± 0.00 |
| DAAO11 | BAC28253.1 | 0.00 ± 0.00 | 0.00 ± 0.00 |
| DAAO12 | BAF32940.1 | 0.00 ± 0.00 | 0.00 ± 0.00 |
| DAAO13 | BAF47961.1 | 0.00 ± 0.00 | 0.00 ± 0.00 |
| DAAO14 | BAF34315.1 | 0.00 ± 0.00 | 0.00 ± 0.00 |
| DAAO15 | BAF34316.1 | 0.00 ± 0.00 | 0.00 ± 0.00 |
| DAAO16 | ALM22233.1 | 0.00 ± 0.00 | 0.00 ± 0.00 |
| DAAO17 | CAJ87425.1 | 0.00 ± 0.00 | 0.00 ± 0.00 |
| DAAO18 | NP_001370668.1 | 0.00 ± 0.00 | 0.00 ± 0.00 |
| DAAO19 | UniProt P80324 | 0.00 ± 0.00 | 0.00 ± 0.00 |
| DAAO20 | CAA90322.1 | 0.00 ± 0.00 | 0.00 ± 0.00 |
| DAAO21 | BAB12222.1 | 0.00 ± 0.00 | 0.00 ± 0.00 |
| DAAO22 | BAA00692.1 | 0.00 ± 0.00 | 0.00 ± 0.00 |
| DAAO23 | EYB24484.1 | 0.00 ± 0.00 | 0.00 ± 0.00 |
| DAAO24 | OBS19408.1 | 0.00 ± 0.00 | 0.00 ± 0.00 |
| DAAO25 | XP_016270247.1 | 0.00 ± 0.00 | 0.00 ± 0.00 |
| DAAO26 | XP_001401161.2 | 0.00 ± 0.00 | 0.00 ± 0.00 |
| DAAO27 | XP_964990 | 0.67 ± 0.05 | 5.56 ± 0.77 |

### 5. Detection of Soluble Expression of D-Amino Acid Oxidase in Genetically Engineered Bacteria

The lysate of genetically engineered bacteria obtained by ultrasonication in step 3 was centrifuged at 12000 rpm, 4° C. for 10 min. 15 μL of the resulting supernatant was mixed well with 3 μL of SDS-PAGE Loading Buffer. After centrifugation, the resulting supernatant was removed, and the resulting precipitate was resuspended in the same volume of buffer. 15 μL of the suspension was taken and mixed well with 3 μL of SDS-PAGE Loading Buffer. The resulting protein samples for electrophoresis were incubated at 99° C. for 10 min.

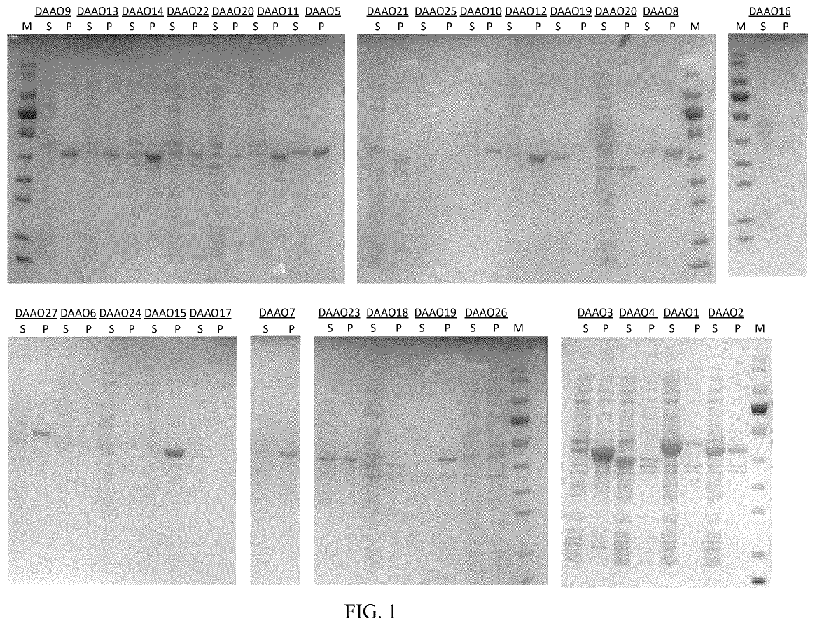

The protein samples (3 μL) were subjected to the protein gel electrophoresis, and the results are shown in FIG. 1. The results of protein gel electrophoresis showed that most of the D-amino acid oxidases exhibited an insoluble expression under the shaking and incubation conditions aforementioned, and a few of them exhibited a good soluble expression, indicating that the soluble expression of D-amino acid oxidase in Escherichia coli needs to be optimized.

### Example 2 Screening of Expression Host for D-Amino Acid Oxidase

Two D-amino acid oxidases, DAAO5 and DAAO27, having enzyme activity on DL-glufosinate as the substrate, were selected. 7 commonly used commercial Escherichia coli expression hosts, including BL21 (DE3), BL21 Star (DE3), Shuffle T7, Tssetta, BL21 (DE3) PLysS, Rosetta-gami 2, and Origami 2 were selected for screening. The pET-28a(+)-DAAO5 and pET-28a(+)-DAAO27 of step 2 in Example 1 were separately transformed into each of 7 aforementioned Escherichia coli hosts by heat stock. The expression of recombinant D-amino acid oxidases was carried out according to the method of step 3 in Example 1. The enzyme activity assay of D-amino acid oxidase was carried out according to the method of step 4 of Example 1, and the results are shown in Table 3. As can be seen from Table 3, DAAO5 had the highest enzyme activity as expressed in Escherichia coli Shuffle T7, followed by in BL21 (DE3), but the difference in enzyme activity was small. DAAO27 had a significantly higher enzyme activity when expressed in BL21 (DE3) compared to other expression hosts. Therefore, BL21 (DE3) was preferred as the expression host for D-amino acid oxidase.

TABLE 3
Enzyme activity assay of D-amino acid oxidase expressed
in different Escherichia coli strains (U/L)

| | DAAO5 | DAAO27 |
|---|---|---|
| BL21(DE3) | 2.93 ± 0.33 | 5.56 ± 0.87 |
| BL21 Star(DE3) | 2.09 ± 0.56 | 1.98 ± 0.06 |
| Shuffle T7 | 3.11 ± 0.28 | 1.72 ± 0.29 |
| Tssetta | 2.07 ± 0.76 | 2.74 ± 0.54 |
| BL21(DE3)PLysS | 1.05 ± 0.89 | 2.59 ± 0.45 |
| Rosetta-gami 2 | 1.86 ± 0.32 | 1.92 ± 0.61 |
| Origami 2 | 1.16 ± 0.22 | 1.03 ± 0.27 |

### Example 3 Addition of Chaperone for Enhancing the Soluble Expression of D-Amino Acid Oxidase

Two D-amino acid oxidases, DAAO5 and DAAO27, having enzyme activity on DL-glufosinate as the substrate, were selected. The purchased Chaperone Plasmid Set (Code No. 3340, TaKaRa) includes 5 different plasmids which express chaperone proteins in Escherichia coli, i.e., PKJE7, PGro7, PTf16, PG-Tf2 and PG-KJE8. The 5 chaperone plasmids were separately constructed into the backbone of pACYC plasmid and then transformed into BL21 (DE3). The pET-28a(+)-DAAO5 or pET-28a(+)-DAAO27 plasmid was transformed into the same BL21 (DE3) cells later.

The successfully constructed engineered bacteria were inoculated and cultured in LB medium. The chaperone protein-expressing plasmids PKJE7, PGro7 and PTf16 were induced by adding 0.5 mg/mL L-arabinose, the chaperone protein-expressing plasmid PG-Tf2 was induced by adding 1 ng/mL tetracycline hydrochloride, the chaperone protein-expressing plasmid PG-KJE8 was induced by adding 0.5 mg/mL L-arabinose in combination with 1 ng/mL tetracycline hydrochloride. These bacteria were cultured in a shaking incubator with a speed of 200 rpm at 37° C. for 2-3 h. When the OD600 value indicating the bacterial density reached 0.8, the bacteria was cooled to a temperature of 18° C. and added with IPTG to a final concentration of 0.5 mM. Then, the triangular flask was transferred to a 18° C. shaking incubator and the bacteria was cultured at 200 rpm for 16 h. After which, the culture was centrifuged at 4000 rpm for 30 min, the supernatant was discarded and the bacterial cells were collected. These cells were then resuspended in 100 mM phosphate buffer (pH 8.0), and broken by ultrasonic treatment to obtain the bacterial suspension as the crude enzyme solution.

The enzyme activity assay of D-amino acid oxidase was performed according to the method of step 4 in Example 1, and the results are shown in Table 4. As can be seen from the results in Table 4, PKJE7 and PTf2 promoted the soluble expression of DAAO5, and PGro7 significantly promoted the soluble expression of DAAO27. Therefore, chaperones PGro7, PKJE7 and PTf2 were preferred to enhance the soluble expression of D-amino acid oxidase in Escherichia coli.

TABLE 4
Enzyme activity assay of D-amino acid oxidase
with addition of chaperone (U/L)

| | DAAO5 | DAAO27 |
|---|---|---|
| None | 2.93 ± 0.33 | 5.56 ± 0.87 |
| PKJE7 | 4.55 ± 1.21 | 2.41 ± 0.74 |
| PGro7 | 3.08 ± 0.41 | 8.07 ± 0.68 |
| PTf16 | 3.22 ± 0.36 | 5.35 ± 0.16 |
| PG-Tf2 | 3.68 ± 0.06 | 2.86 ± 0.09 |
| PG-KJE8 | 1.74 ± 0.18 | 1.26 ± 0.21 |

### Example 4 Effect of PGro7 Chaperone on DAAO27 Expression in One or Two Plasmids

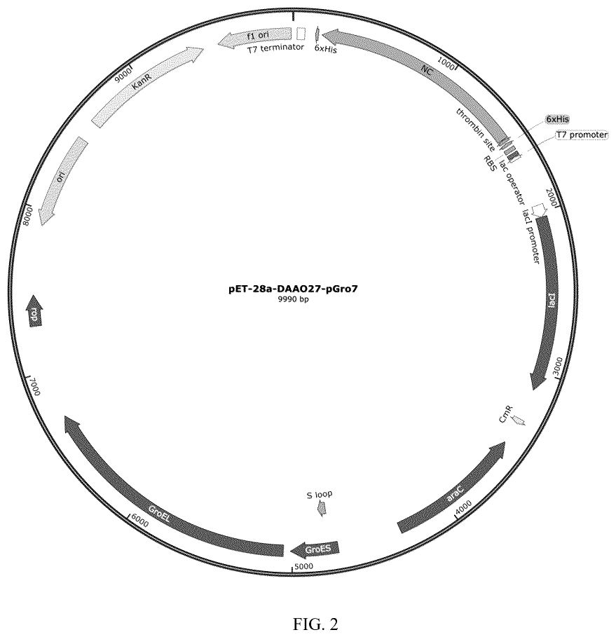

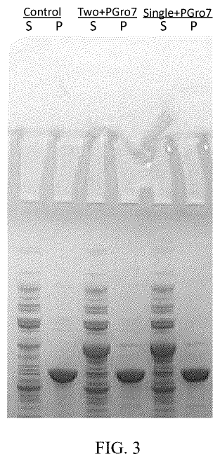

To further investigate the effect of the expression form of PGro7 chaperone on D-amino acid oxidase 27 (DAAO27), a single plasmid co-expressing PGro7 and DAAO27 was constructed in pET28a plasmid vector and the chaperone coding sequence was inserted between the replicon and the LacI repressor of pET28a (FIG. 2). Genetically engineered bacteria containing two plasmids independently expressing PGro7 and DAAO27 were constructed. The expression and enzyme activity assay of recombinant DAAO27 were carried out according to the induced expression process in Example 3, and the results of the assay are shown in Table 5 and FIG. 3. As can be seen from Table 5, the expression of the PGro7 chaperone improved the enzyme activity of D-amino acid oxidase, and the co-expression of PGro7 and DAAO27 in one plasmid had a more significant promotion effect on enzyme activity, compared with the separate expression of the two.

TABLE 5
Enzyme activity assay of D-amino acid oxidase co-expressed
PGro7 chaperone in one or two plasmids (U/L)

| | DAAO27 |
|---|---|
| None | 5.56 ± 0.87 |
| Expression of two plasmids | 8.99 ± 0.32 |
| Expression of single plasmid | 9.59 ± 0.67 |

### Example 5 Substrate Spectrum of D-Amino Acid Oxidase

D-Ala, D-Asp, D-Glu, D-Ser, D-Nva, D-Phe, and D-Hpa were selected as substrates to determine the enzyme activities of DAAO1, DAAO2, DAAO3, DAAO4, DAAO5 and DAAO27 on D-amino acids, and the results are shown in the table below. As can be seen from Table 6, the substrate spectra of the three D-amino acid oxidases DAAO1, DAAO2 and DAAO4 were broad, they had catalytic activity on 6-7 kinds of D-amino acids aforementioned, whereas the substrate spectra of DAAO3, DAAO5 and DAAO27 were narrow, and they had catalytic activity on only 1-2 kinds of D-amino acids.

TABLE 6
Substrate spectrum of D-amino acid oxidase (U/g cell dry weight)

| Number of | | | | | | | |
|---|---|---|---|---|---|---|---|
| D-amino acid | | | | | | | |
| oxidase | D-Ala | D-Asp | D-Glu | D-Ser | D-Nva | D-Phe | D-Hpa |
| DAAO1 | 16.79 ± 0.02 | 22.27 ± 0.21 | 0.63 ± 0.21 | 12.15 ± 0.34 | 14.07 ± 0.37 | 9.27 ± 0.46 | 27.06 ± 0.46 |
| DAAO2 | 1.52 ± 0.18 | 18.56 ± 0.14 | 7.67 ± 0.32 | 0.45 ± 0.17 | 1.37 ± 0.22 | 1.74 ± 0.05 | 0.00 ± 0.21 |
| DAAO3 | 0.00 ± 0.00 | 4.34 ± 0.11 | 0.00 ± 0.00 | 0.00 ± 0.00 | 0.00 ± 0.15 | 0.00 ± 0.32 | 0.00 ± 0.14 |
| DAAO4 | 15.14 ± 0.08 | 12.06 ± 0.08 | 0.00 ± 0.12 | 2.40 ± 0.09 | 20.22 ± 0.43 | 9.47 ± −0.56 | 2.83 ± 0.65 |
| DAAO5 | 0.00 ± 0.00 | 0.00 ± 0.03 | 9.90 ± 0.13 | 0.00 ± 0.22 | 0.00 ± 0.00 | 0.00 ± 0.00 | 0.00 ± 0.34 |
| DAAO27 | 0.00 ± 0.00 | 1.77 ± 0.12 | 1.20 ± 0.05 | 0.00 ± 0.09 | 0.00 ± 0.00 | 0.00 ± 0.00 | 0.00 ± 0.06 |

The above embodiments are only preferred embodiments of the present disclosure. It should be noted that, for those skilled in the art, several improvements and modifications may be further made without departing from the principle of the present disclosure, and these improvements and modifications should also be deemed as falling into the protection scope of the present disclosure.

1-15. (canceled)

16. A method for producing α-keto acid, comprising contacting a D-amino acid oxidase with a D-amino acid or a racemic amino acid and conducting an oxidation reaction, and the D-amino acid oxidase is selected from a D-amino acid oxidase encoded by a nucleic acid selected from any one of SEQ ID NOs: 1-6.

17. The method according to claim 16, wherein the D-amino acid oxidase is encoded by SEQ ID NO: 6.

18. The method according to claim 16, wherein the D-amino acid oxidase is produced by Escherichia coli.

19. The method according to claim 18, wherein an expression cassette is integrated into the genome of the Escherichia coli or the Escherichia coli contains a plasmid carrying an expression cassette, and the expression cassette comprises SEQ ID NO: 6.

20. The method according to claim 19, wherein the expression cassette further comprises a promoter and a terminator.

21. The method according to claim 20, wherein the promoter is selected from the group consisting of T7 promoter, sCMV promoter, Lac promoter, tac promoter, IPL promoter, araB promoter, Pzt-1 promoter, and trp promoter.

22. The method according to claim 20, wherein the terminator is selected from the group consisting of T7 terminator, rrnB T1 terminator, p-independent terminator, and p-dependent terminator.

23. The method according to claim 18, wherein the Escherichia coli further comprises a nucleic acid encoding a molecular chaperone, and the molecular chaperone is selected from the group consisting of groES, groEL, dnaK, dnaJ, tig, and grpE.

24. The method according to claim 23, wherein the nucleic acid encoding the molecular chaperone is integrated into the genome of the Escherichia coli, or an expression cassette containing the nucleic acid encoding the molecular chaperone is transformed into the Escherichia coli.

25. The method according to claim 23, wherein the nucleic acid encoding the molecular chaperone and the nucleic acid encoding the D-amino acid oxidase are in one vector or two vectors.

26. The method according to claim 18, wherein the Escherichia coli is selected from the group consisting of BL21 (DE3), BL21 (DE3) Star, Shuffle T7, Tssetta, BL21 (DE3) Plyss, Rosetta-gami 2, and Origami 2.

27. The method according to claim 26, wherein the Escherichia coli is BL21 (DE3).

28. The method according to claim 16, wherein the D-amino acid is D-phosphinothricin and the racemic amino acid is DL-phosphinothricin.

29. The method according to claim 16, comprising:
integrating the nucleic acid encoding the D-amino acid oxidase into the genome of an Escherichia coli, or transforming an expression cassette containing a nucleic acid encoding the D-amino acid oxidase into an Escherichia coli, wherein the nucleic acid encoding the D-amino acid oxidase is selected from SEQ ID NOs: 1-6;
culturing the Escherichia coli;
collecting and sonicating the cells to obtain a suspension containing the D-amino acid oxidase, which is a crude enzyme solution;
contacting the crude enzyme solution with a substrate to conduct an oxidation reaction, wherein the substrate is a D-amino acid or a racemic amino acid.

30. The method according to claim 29, wherein the Escherichia coli further expresses a molecular chaperone and the molecular chaperone is co-expressed with the D-amino acid oxidase, and wherein the molecular chaperone is selected from the group consisting of groES, groEL, dnaK, dnaJ, tig, and grpE.

31. A method for producing a D-amino acid oxidase with improved enzymatic activity, comprising fermenting an Escherichia coli containing a nucleic acid encoding the D-amino acid oxidase, wherein the nucleic acid encoding the D-amino acid oxidase is selected from any one of SEQ ID NOs: 1-6.

32. The method according to claim 31, wherein the nucleic acid encoding the D-amino acid oxidase is integrated into the genome of the Escherichia coli, or an expression cassette containing the nucleic acid encoding the D-amino acid oxidase is transformed into the Escherichia coli.

33. The method according to claim 31, wherein the Escherichia coli further expresses a molecular chaperone and the molecular chaperone is co-expressed with the D-amino acid oxidase, and wherein the molecular chaperone is selected from the group consisting of groES, groEL, dnaK, dnaJ, tig, and grpE.

34. The method according to claim 33, wherein the molecular chaperone is encoded by a nucleic acid and the nucleic acid is integrated into the genome of the Escherichia coli, or an expression cassette containing the nucleic acid encoding the molecular chaperone is transformed into the Escherichia coli.

35. The method according to claim 33, wherein the nucleic acid encoding the molecular chaperone and the nucleic acid encoding the D-amino acid oxidase are in one vector or two vectors.
